(19) United States
(12) Patent Application Publication
(10) Pub. No.: US 2019/0254596 A1
(43) Pub. Date: Aug. 22, 2019

(54) METHOD FOR GENERATING A MAGNETIC RESONANCE IMAGE DATASET, COMPUTER PROGRAM PRODUCT, DATA MEDIUM, AND MAGNETIC RESONANCE SYSTEM

(71) Applicant: Siemens Healthcare GmbH, Erlangen (DE)

(72) Inventors: Marcel Dominik Nickel, Herzogenaurach (DE); Manuel Schneider, Erlangen (DE)

(21) Appl. No.: 16/272,231

(22) Filed: Feb. 11, 2019

(30) Foreign Application Priority Data
Feb. 20, 2018 (DE) 10 2018 202 546.0

(51) Int. Cl. A61B 5/00; G01R 33/565; G01R 33/561; G01R 33/46; G01R 33/54

(52) U.S. Cl. CPC A61B 5/4872; G01R 33/565; G01R 33/543; G01R 33/4625; G01R 33/5616

(57) ABSTRACT

A method is provided for generating a magnetic resonance image dataset containing spectroscopic information from an in vivo measurement. The method includes acquiring measurement data by a non-Cartesian k-space sampling scheme, performing a gradient correction of the measurement data, regridding the measurement data to Cartesian coordinates, Fourier-transforming the measurement data, determining the fat component and/or the fatty acid components of at least some of the volume elements using a model function applied to the signal distribution, and generating at least one magnetic resonance image dataset in which one piece of the spectroscopic information is mapped in a spatially resolved manner. A computer program product, a data medium, and a magnetic resonance system are also disclosed.

The present patent document claims the benefit of German Patent Application No. DE 10 2018 202 546.0, filed Feb. 20, 2018, which is also hereby incorporated by reference.

### TECHNICAL FIELD

The disclosure relates to a method for generating a magnetic resonance image dataset containing spectroscopic information from an in vivo measurement.

### BACKGROUND

In order to visualize the spatial distribution of different substances or tissues or spin species, it is known to make use of the phenomenon known as chemical shift. This entails supplementing a spectroscopic experiment with phase encoding gradients in order to generate spatial information. These experiments are summarized under the acronym CSI, for chemical shift imaging.

Due to the use of phase gradients in all spatial directions, these methods are very time-intensive.

It is furthermore known to exploit the different phase shifts of fat and water protons in order to overlay the spins additively and subtractively in order to determine a separate water image and a separate fat image from two or three overlay images. These methods are known as 2-point Dixon or 3-point Dixon methods.

Dixon methods are known from U.S. Patent Application Publication No. 2017/0146624 A1, U.S. Patent Application Publication No. 2017/0082709 A1, and U.S. Patent Application Publication No. 2015/0061667 A1, for example.

Spin echo (SE) sequences may be used in this case. Fast spin echo (FSE) denotes a technique in which a plurality of spin echoes is acquired in a train. In addition, a gradient echo train is generated for each spin echo by way of bipolar gradients. This train may contain two, three, or more gradient echoes. One of the gradient echoes is brought into congruence with the spin echo in the echo train, the echo being the at least one echo with additive overlaying: the water and fat spins are “in phase”. The gradient echo before and the gradient echo after are placed in such a way that the spins are disposed in an “opposed phase” arrangement.

With this approach, it is possible to represent the fat distribution. However, it is not possible in this case to differentiate between the individual components of the fat signal.

Fats are esters of glycerol. The esterification takes place with monocarbon acids. As glycerol is trivalent, fats have three fatty acids.

The physical properties of the fats are dependent on the chain length (cl) and the frequency of the C═C double bonds in the fatty acid residues. These also determine the nomenclature. Singly unsaturated (monounsaturated) fatty acids have one double bond, whereas multiply unsaturated (polyunsaturated) fatty acids have a plurality of double bonds. Fatty acids without C═C double bond are called saturated fatty acids.

The resonance frequency of the protons in the fatty acids is influenced here not only by the chain length and the number of double bonds (ndb), but also by the number of double bonds interrupted by a methylene group (e.g., number of methylene-interrupted double bonds or “nmidb”). Methylene groups may separate two double bonds from one another.

In the human body, fats are deposited in adipose or fat cells. These represent a form of the connective tissue. Fat cells in this case accumulate fats for different reasons. In addition to food intake, diseases may also play a role.

In the liver, fat is stored in the liver cells. One disease of the liver is the condition known as fatty liver. A fatty liver may be brought about by overeating, diseases, alcohol abuse, or other causes. One group of diseases are the so-called non-alcoholic fatty liver diseases (NAFLDs). The symptoms range from the simple accumulation of fat in the liver through to fatty liver cirrhosis. Due to an increasing spread of risk factors such as type 2 diabetes, increasing numbers of fatty liver cirrhosis, and consequently increasing numbers of organ transplants, are likely.

Moreover, there are not only different stages but also different types of non-alcoholic fatty liver diseases. These may be differentiated on the basis of the different degree of saturation of individual fatty acids.

The gold standard for the diagnosis of a non-alcoholic fatty liver disease is in this case realized in the form of an invasive biopsy. As well as general biopsy risks, this has the disadvantage that the result may be distorted by the local sample taking. A non-alcoholic fatty liver disease may therefore be wrongly detected or rejected by mistake.

In certain questions, it is however vital to know not only the total fat content but also the fatty acid composition. This applies as much to non-alcoholic fatty liver disease as to coronary diseases.

In order to be able also to carry out repeated examinations on patients, there is therefore a need for a measurement method which is non-invasive, which permits a differentiation of the fatty acid composition, and which at the same time may also be used in vivo without additional overhead. Because it is intended to be applied to patients, it is also desirable that no breath-holding is necessary.

A method for determining the fat content and the fatty acid composition in an examination subject is known from Peterson P, Minsson S.: Simultaneous quantification of fat content and fatty acid composition using MR imaging, Magn Reson Med. 2013; 69(3):688-697. The measurement data is obtained in this case by a standard Cartesian sampling scheme.

### SUMMARY AND DESCRIPTION

The scope of the present disclosure is defined solely by the appended claims and is not affected to any degree by the statements within this summary. The present embodiments may obviate one or more of the drawbacks or limitations in the related art.

It is an object of the present disclosure to generate a magnetic resonance image dataset which permits a spatially resolved determination of the fatty acid composition in an examination region or examination volume and in which the quantification satisfies medical questions.

This object is achieved by a method for generating a magnetic resonance image dataset containing spectroscopic information from an in vivo measurement. The method includes: acquiring measurement data by a non-Cartesian k-space sampling scheme, wherein measurement data is acquired at different echo times in order to measure a time-dependent signal waveform; performing a measurement data correction to eliminate gradient errors; regridding the measurement data to Cartesian coordinates; Fourier-transforming the measurement data to produce an image dataset containing image elements; determining at least one piece of spectroscopic information in the form of the fat component and/or at least one fatty acid component of at least some of the image elements using a model function applied to the signal waveform; and generating at least one magnetic resonance image dataset in which one piece of the spectroscopic information is mapped in a spatially resolved manner.

The method may include a sequence of acts beginning with the acquisition of the measurement data up to and including the generation of the fat distribution or component image or images which permit an in vivo experiment and ultimately also culminate in a medically valuable conclusion.

The acquisition of the measurement data was carried out here using a non-Cartesian sampling scheme in order to enable a measurement to be performed without breath-holding. Known methods of non-Cartesian sampling are radial or spiral sampling. Measurement data is in this case all data used to generate the fat images. The measurement data may be present as echo signals or FIDs. It may have been acquired by parallel imaging, (e.g., using a plurality of coils simultaneously), or by a single receive coil. Basically, the data may be in the form of simple 2D, multi-slice 2D or 3D image datasets.

Inaccuracies in the assignment of the measurement data to the corresponding k-space positions may result due to systematic gradient errors. A correction of gradient errors may therefore be performed on the measurement data. This is possible in different ways and is explained in more detail further below.

In order to be able to Fourier-transform the spiral-shaped or radial k-space data into an image dataset, in particular using the fast Fourier transform FFT, it is necessary to transfer the k-space data into a Cartesian grid. This process is known as gridding or regridding. In gridding, the Cartesian k-space points are obtained according to the following formula:

M_{cart}(x,y)={[M·S·S]⊕C}·K⊕⁻¹C

where “M” denotes the magnetization of the k-space, “S” the acquisition coordinates, “W” a weighting function, “C” a convolution function, and “K” the Cartesian grid.

Finally, the data of the Cartesian k-space is obtained here by weighting and interpolation of the neighboring acquired data points.

The weighting function specifies how the measured k-space data is incorporated into the calculated data in order thus to compensate for the varying sampling density. The weighting function may be obtained from the sampling coordinates “S” and the convolution function “C”.

For the interpolation, in contrast, a convolution is performed using a window function. A known window function is the Hamming window. This is to be chosen such that on the one hand the Cartesian grid has no gaps and on the other hand also not too distant data points are taken into account.

The projection is thus yielded as:

M_{cart}(x,y)=M_{conv}(x,y)*K(x,y)

Once all the gridding acts have been completed, a k-space is obtained having calculated or transformed Cartesian data points instead of radially or spirally distributed data points. These may then be processed further using known postprocessing acts such as zero filling, FFT, etc.

Complex-valued numbers are obtained by the subsequent Fourier transform, the respective representation as real and imaginary part or as magnitude and phase being equivalent. Irrespective of the representation, these represent an image dataset. This is populated with complex-valued numbers.

The numbers of the image dataset are then fitted by a model function. As also in the determination of the relaxation times, this process may entail proceeding image element by image element. In the case of 2D datasets, this is then referred to as pixel-wise fitting, and in the case of 3D datasets as voxel-wise fitting. The model function in this case maps the signal waveform. In the fitting process, the variable parameters are determined in such a way that the total deviation of the model function from the measurement data is minimized. Parameter maps are obtained as result.

Not all image elements must be fitted in this process. With the aid of a threshold value, (e.g., for the SNR), image elements having only noise signal may be omitted.

A predefinable examination region may be selected by pattern recognition. If only the fat distribution in the liver is relevant, the image elements that image the liver may be marked by pattern recognition as ROI (region of interest) or VOI (volume of interest). This may also only be achieved in certain cases, e.g., during an examination with a patient in the magnetic resonance system. In this case, in order to lose no time during the measurement data processing and to be able to start, e.g., follow-on measurements promptly, it is possible to evaluate only the liver but not the surrounding tissue. The omitted tissue may be postprocessed either during the next measurement or when considering the reconstructed partial images, or even never.

A radial sampling scheme may advantageously be used for acquiring the measurement data. In relation to the measurement speed and the subsequent ability to eliminate gradient errors, this has proven to be the most advantageous sampling technique. Trajectories describe the position of the read direction and in this case lie at an acquisition angle referred to an output direction. This is used as information during the regridding.

Measurement data may be acquired from at least 200 different acquisition angles. In the case of a gradient echo having one echo signal per excitation cycle and one slice, there may be 200 or more echo signals, each echo signal having been captured at a different acquisition angle. In the case of a multigradient echo, a corresponding multiple of echo signals is produced.

A method for acquiring a magnetic resonance dataset, also known as a measurement sequence, may include three phases: excitation phase, evolution phase, and detection phase.

Preparation modules may be used during the excitation phase. This concludes with the last RF excitation pulse. In the case of a gradient echo, the excitation phase includes only the 90° pulse, while in the case of a FLASH, the excitation phase includes the RF pulse at a smaller flip angle. The deflection angle of the RF pulses is referred to as the flip angle in order to avoid confusion with the acquisition angle of the echo signals in the radial sampling approach.

The excitation phase may entail applying an excitation pulse, in particular a single excitation pulse. The excitation pulse may advantageously be embodied as a multiband pulse, in particular as a dual-band pulse. The excitation pulse may then excite two or more slices simultaneously. A slice selection gradient may be applied simultaneously during the application of the excitation pulse. This is possible irrespective of the pulse shape.

Slices that are acquired simultaneously lie parallel to one another, i.e., they do not intersect one another.

The evolution phase may contain at least one slice rephasing gradient and/or at least one read dephasing gradient.

In the detection phase, read gradients may be applied in imaging sequences and the acquisition window is also open.

In a sequence, an excitation phase may also be followed by a plurality of evolution and detection phases, as is the case, e.g., in FSE. Otherwise, the phases are repeated until all measurement signals have been acquired. In this context, this means until acquisition passes have been executed at all acquisition angles.

The sequence of acts may also predefine the execution sequence with respect to time. In other words, the measurement data is first corrected with calibration data before it is Fourier-transformed. That said, however, the correction may in principle be carried out “on the fly”, e.g., during the measurement or after the measurement.

Because the method disclosed herein may be used in different measurement sequences, the following definitions are presented.

A measurement sequence refers as in the conventional sense to a sequence of RF pulses, gradient fields, latency times, and acquisition windows which precisely specify and characterize the order of execution of the measurement sequence. Examples of measurement sequences are the already cited FLASH, gradient echo, EPI, TrueFisp, etc. The image datasets determined therefrom may also include weightings by preparation modules or be suitable for generating maps such as T1 maps or T2 maps.

In this case, a measurement sequence includes defined or definable partial experiments. A partial experiment may also be referred to as an excitation cycle. In a radial sampling scheme, a partial experiment is an acquisition at precisely one acquisition angle. An excitation cycle is one repetition time TR long.

Measurement data may advantageously be acquired from at least 400 different acquisition angles. Measurement data may furthermore be acquired from at least 600 different acquisition angles. Measurement data may advantageously be acquired from at least 800 different acquisition angles.

A multigradient echo may be used for the measurement data acquisition.

Although fast spin echo sequences may be employed in known Dixon methods, it is now proposed to use a multigradient echo.

This means that a plurality of echo signals are acquired as measurement data at each acquisition angle. Basically, these echo signals may also be used for determining the T₂* relaxation. It should however be noted here that multiple spin species influence the signal waveform.

The excitation pulse used may have a flip angle of less than 90°, less than 20°, or less than 10°. The flip angle may be 4°. The measurement time may be minimized as a result.

The repetition time may be less than 100 ms, less than 50 ms, or less than 20 ms. The measurement time may be minimized as a result.

The read gradients may advantageously be embodied as bipolar gradients. Minimum echo times may be realized as a result.

Advantageously, at least 12 echo signals, in particular, precisely 12 echo signals, may be acquired in one detection phase or in one gradient echo train. Alternatively, precisely 16 echo signals may be acquired. With this number, the usefulness of the data in a medical context is assured, e.g., the standard deviation of the results is sufficiently small. On the other hand, the volume of the measurement data, and consequently also the postprocessing time, may be kept as small as possible.

After or during the acquisition of the measurement data, gradient errors contained in the measurement data may be eliminated as far as possible. Such errors may be caused by eddy currents. The data used to correct gradient errors in the measurement data is referred to hereinbelow as calibration data.

The calibration data may be acquired in a separate measurement. Firstly, the processing of the data is then simplified because measurement data or image data and calibration data do not need to be separated, but are available already separated. Secondly, the calibration measurement may then remain limited to the necessary minimum and is not determined by the parameters of the imaging data.

In order to obtain calibration data for performing the measurement data correction, calibration measurement data may be acquired using the same measurement sequence. If a multigradient echo with a repetition time TR and echo times TE1, TE2, . . . is used for the acquisition, then the measurement sequence is a multigradient echo with a repetition time TR and echo times TE1, TE2, . . . . The parameters that determine the image contrast in relation to the relaxation times are therefore identical.

A difference may however be made in the parameter or parameters that determine the resolution. The resolution of the calibration measurement dataset may be lower than that of the image measurement dataset. For example, the number of acquisition angles in the calibration measurement may be less than in the case of the image measurement. In this case, the measurement data of the calibration measurement is also echo signals. The difference therefore lies not in the type of data, but in the way the data is used.

In an alternative embodiment, a portion of the measurement data is used for the calibration. The measurement sequence, (e.g., its acquisition angle), may then be planned so that measurement data with which a calibration may be performed is available.

The calibration data may include at least one pair of measurement data acquired at opposite acquisition angles. Opposite acquisition angles are an acquisition angle and the acquisition angle +180°. A pair of opposite acquisition angles is therefore e.g., 0° and 180°; 90° and 270°; 45° and 225°; or 135° and 315°.

Advantageously, precisely four acquisition angles may be used during the acquisition of the calibration measurement data by a radial sampling of the k-space. In particular, precisely two pairs of measurement data may be acquired at opposite angles. These may be arranged at right angles to one another. A possible angle combination includes, for example, the acquisition angles 0°, 90°, 180°, and 270°.

The number of acquisition angles actually to be used is therefore at a fraction of the data required for the imaging.

Because the acquisition angle specification for the calibration data is not necessarily fulfilled during the acquisition of the image data, a separation of the measurements is advantageous because the calibration data may be acquired in minimum time.

The determination of the correction values then results as follows.

From the measurement data of the first pair acquired at opposite acquisition angles, a Δk₁(n) is determined for each echo by calculating the distance between the signal maxima. The index n in this case stands for the number of the echo.

This is also accomplished with the second pair of calibration data acquired at opposite acquisition angles, yielding a Δk₂(n).

Because Δk₁(n) and Δk₂(n) stand perpendicularly on one another, a Δk(n) may likewise be determined at every other acquisition angle by the formula:

Δk_{Θ}(n)=Δk₁(cos Θ)²+Δk₂(sin Θ)²

where Θ is the acquisition angle, which is now expressed as a function of the measurement direction of the calibration data. If the measurement directions of the calibration data lie in the x- and y-direction, the result is yielded by:

Δk_{Θ}(n)=Δkₓ(cos θ)²(n)+Δk_{y}(sin θ)²(n)

Because the reference direction may change during this process, different acquisition angle designations Θ and θ have been used.

Thus, a separate calibration value is obtained for each echo.

A separate calibration value may be determined in addition for each (receive) channel. A separate calibration value then exists for each echo as a function of the acquisition angle, the echo number and the channel. If there are multiple slices or partitions, the calibration values may also be determined separately for each slice or partition.

Following the correction of the measurement data, the obtained signals, (e.g., the complex-valued measurement data), may be fitted image element by image element.

In this case, the fat component and/or the fatty acid components may be taken into account in the model function. In particular, the phase shift of individual elements of the fatty acids may be taken into account.

The following relation may be used as the model function:

D(tₙ)=(W+FfΣₘ₌₁⁹αₘ(ndb,nmidb,cl)·e^{i2πΔf}^{ₘ}^{t}^{ₙ})e^{i2πψt}^{ₙ}e^{-R}^{₂}^{^{*}}^{t}^{ₙ}

where:

W denotes the water signal,

F denotes the fat component,

αₘ denotes the relative amplitude of the m^{th} fat peak,

Δfₘ denotes the frequency shift of the m^{th} fat peak,

f denotes a normalization factor,

Ψdenotes the off resonance,

R₂* denotes the transverse relaxation rate, and

tₙ denotes the echo time of the nth echo.

This model function is fitted pixel by pixel to the measurement data. Given 12 gradient echoes, there exist 12 complex-valued numbers for one image element, e.g., 24 numeric values. The model function is configured to these in order to minimize the deviation overall. Parameter maps are obtained as result. With the formula given above, there results a map for the signal intensity of the water, a map for the signal intensity of the fat, and a separate map for each of the m fatty acid peaks. Possible maps include a map charting saturated fatty acids, a map charting monounsaturated fatty acids, and a map charting polyunsaturated fatty acids. In an advantageous embodiment variant, the maps for ndb, nmidb and cl are calculated, and then maps for saturated/monounsaturated/polyunsaturated maps are determined therefrom.

Irrespective of other values, it is initially assumed in this case that the fat signal has 9 peaks. Alternatively, however, it is also possible to use 3 or 4 peaks as a basis in the model function. In the summation function, the sum then runs from m=1 to m=3 or m=4. In other words, the fatty acid components are determined using a spectral modeling scheme which employs a linear superposition with more than two basis vectors. In certain examples, the linear superpositions have three or four basis vectors.

The resonance shift is then taken into account in the determination of the fat component and/or the fatty acid components.

The method may advantageously be applied to measurement data that images a liver or a part of a liver as the organ under examination. An application case may then serve for establishing the fatty acid distribution in order to determine the presence of the type of a non-alcoholic fatty liver disease.

The object cited in the introduction is also achieved by a computer program product or computer program which may be used for controlling a control device which controls an image generation unit of a magnetic resonance system which performs the aforesaid method.

In addition, the disclosure relates to a data medium for a control device for controlling a data generation unit of a magnetic resonance system containing data for performing the described method. The data generation unit may advantageously be an image generation unit.

The disclosure further relates to a magnetic resonance system including a control device. The magnetic resonance system is characterized in that the control device is embodied for performing the method as described.

The implementation of the aforesaid methods in the control device may in this case be accomplished in the form of software or alternatively also as (e.g., permanently wired) hardware.

Further advantageous embodiments of the magnetic resonance system correspond to equivalent embodiments of the method. In order to avoid unnecessary repetitions, reference is therefore made to the corresponding method features and their advantages.

### BRIEF DESCRIPTION OF THE DRAWINGS

Further advantages, features, and specifics of the present disclosure will become apparent from the following description of advantageous embodiments.

FIG. 1 depicts an example of a magnetic resonance system.

FIG. 2 depicts an example of a measurement sequence.

FIG. 3 depicts a k-space in a first representation.

FIG. 4 depicts a k-space in a second representation.

FIG. 5 depicts a k-space in a third representation.

FIG. 6 depicts an example of a magnetic resonance image dataset.

### DETAILED DESCRIPTION

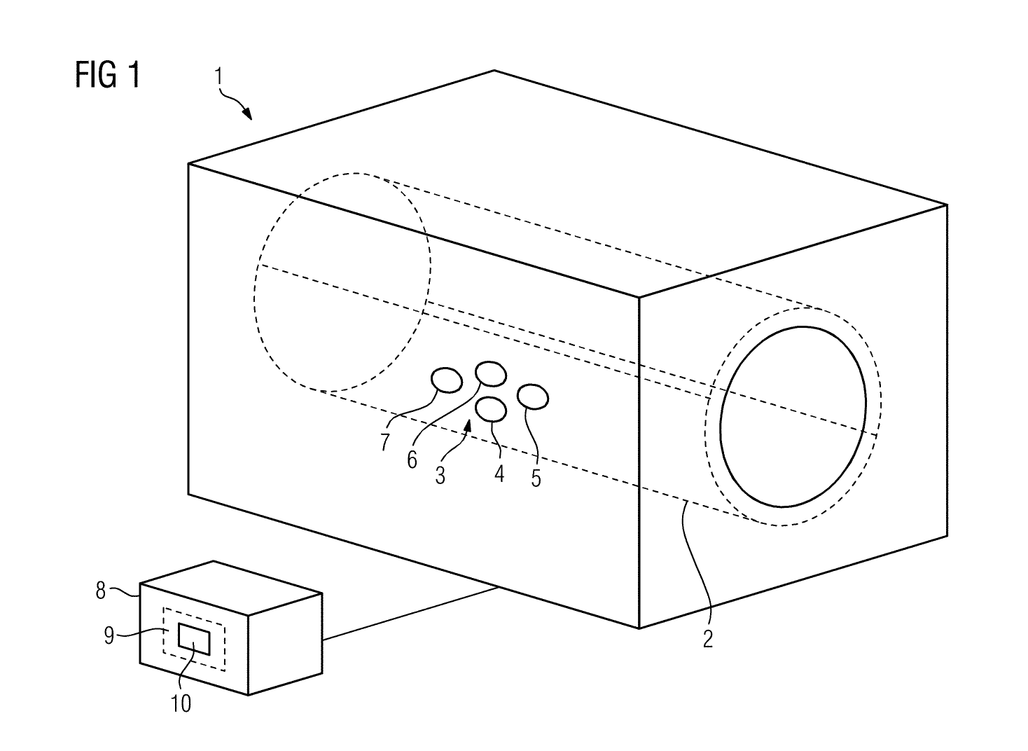

FIG. 1 depicts a magnetic resonance system 1 having a transmit coil arrangement 2. The transmit coil arrangement 2 may be embodied as a body coil. However, it may also be a transmit coil array in this case. The transmit coil arrangement 2 is represented by a dashed outline.

For data acquisition purposes, the magnetic resonance system 1 possesses a receive coil arrangement 3. The receive coil arrangement 3 may be a coil array including coils 4, 5, 6, and 7. The coils 4, 5, 6, and 7 read out measurement signals simultaneously, and therefore in parallel.

The magnetic resonance system 1 has a control device 8 for controlling the experiments.

The magnetic resonance system 1 additionally possesses, as part of the control device 8 or independently thereof, a data medium 9 on which computer programs 10 for performing magnetic resonance measurements are stored.

For clarity of illustration reasons, other components of the magnetic resonance system 1, such as gradient coils or a patient couch, are not shown.

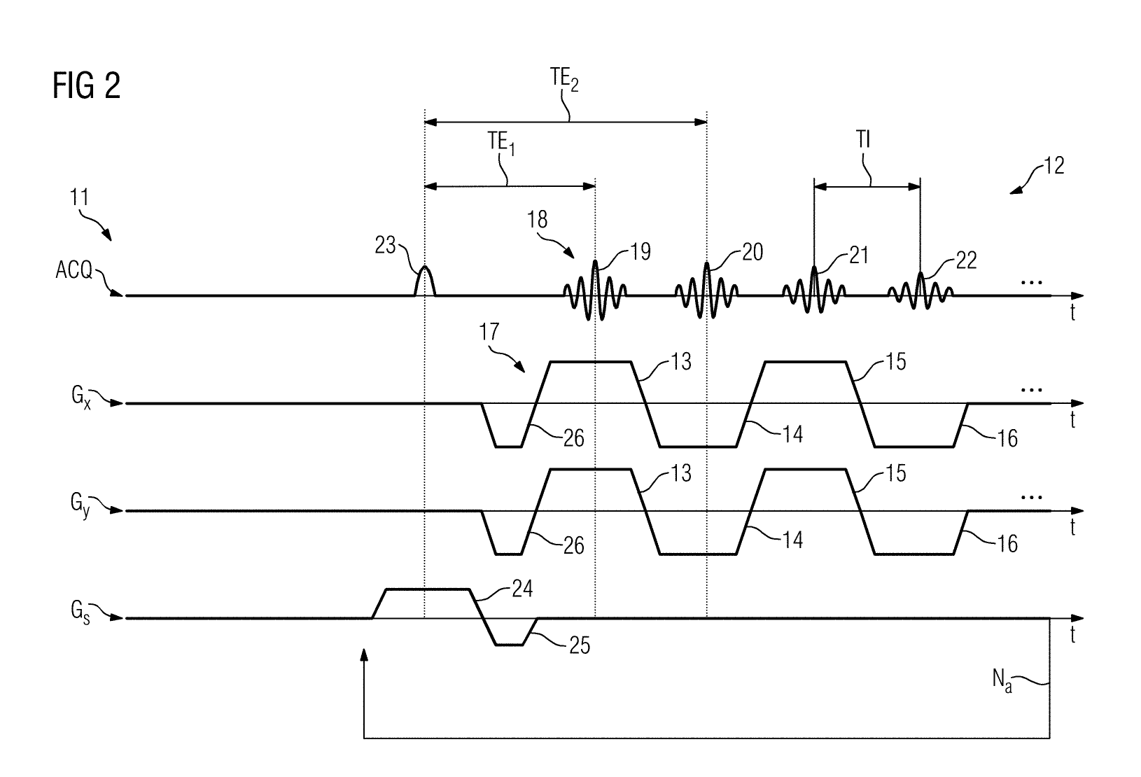

FIG. 2 depicts a sequence diagram 11 of a multigradient echo measurement sequence 12 using a radial k-space sampling scheme. The radial sampling is distinguishable to the extent that the directions Gₓ and G_{y} are specified instead of the read direction G_{R} and the phase direction G_{P}. There is also no phase encoding present. The gradient strengths in the directions Gₓ and G_{y} are chosen such that the acquisition is performed at a thus resulting acquisition angle.

The read gradients 13, 14, 15 and 16 are represented by way of example as read gradients of a read gradient train 17 for generating a gradient echo train 18 containing echo signals 19, 20, 21 and 22. As described further above, twelve or more echo signals may be measured, as is indicated by the three dots. A corresponding number of read gradients may be used.

The echo signals 19, 20, 21 and 22 have different echo times TE₁, TE₂, . . . . Their signal intensities decrease with T₂*.

The multigradient echo 12 has a slice-selective excitation pulse 23. A slice selection gradient 24 is applied simultaneously therewith. This is followed by a slice refocusing gradient 25 in the slice selection direction G_{S}.

In an embodiment, the excitation pulse 23 may be a dual-band pulse. The flip angle of the excitation pulse 23 may be a few degrees, (e.g., 4°). The repetition time may then be chosen under 20 ms. At an interecho time of 1.2 ms, twelve echo signals may be acquired.

If a 3D experiment is to be performed instead of a multi-slice experiment, a gradient having variable strength is applied instead of the constant slice selection refocusing gradient 25 in order to impress a phase encoding in the slice selection direction. The Fourier transform may then also be performed in three spatial directions. The individual planes are then also referred to as partitions. In a multi-slice experiment, the planes are called slices.

A read dephasing gradient 26 is applied before the read gradient train 17.

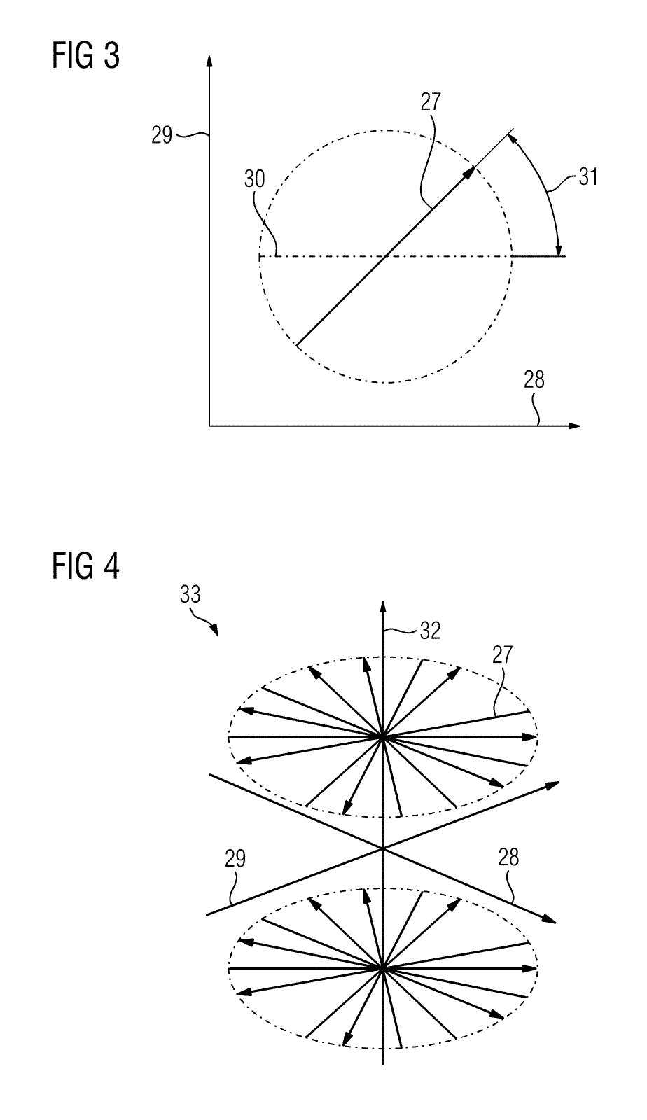

FIG. 3 depicts a two-dimensional representation of a k-space. A single trajectory 27 is shown therein.

The axis 28 denotes the kₓ direction, and the axis 29 the k_{y} direction. Between the line 30 drawn as parallel to the kₓ axis and the trajectory 27 there lies the acquisition angle 31. This is designated by “0” in the formulae given further above. The critical factor here is that the same reference axis is used in each case, (e.g., the same reference direction is chosen), for the regridding or for the fitting of the model function.

FIG. 4 depicts the k-space for an MR experiment using two slices or two partitions. The axis 32 denotes the k_{z} direction. A plurality of trajectories 27 is acquired in each slice or partition. As already described above, between 200 and 800 trajectories 27 may be acquired in each slice or partition. The acquisition angles 31 in this case cover all spatial directions. Averaging operations are not included in the number of acquisition angles. These may increase the number of measurements by a multiple in each case.

The totality of the measurement data acquired along the trajectories 27 then produces a measurement dataset 33.

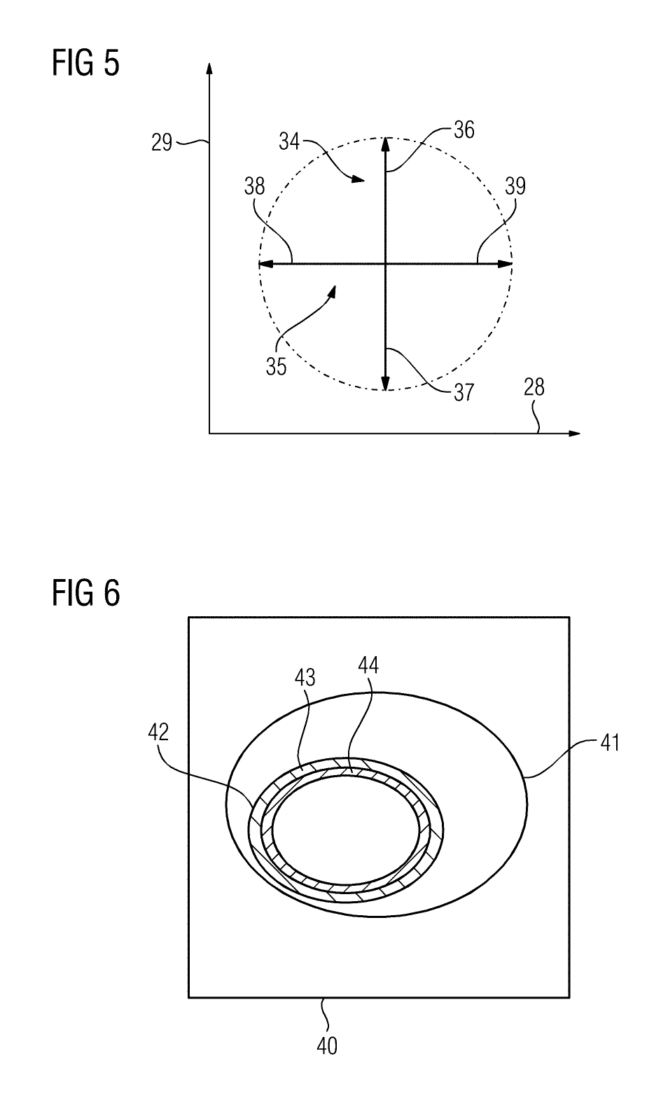

FIG. 5 depicts a possible k-space sampling scheme for the acquisition of calibration data. In this case, two pairs 34 and 35 of trajectories 36 and 37 and 38 and 39, respectively, are present. The trajectories 36 and 37 have opposite acquisition angles. Starting from the kₓ axis 28, the trajectory 36 has an angle of 90°, and the trajectory 37 has an angle of 270°. The trajectories 38 and 39 stand perpendicularly thereon. The trajectory 38 possesses an acquisition angle of 180°, and the trajectory 39 an acquisition angle of 0°.

As described further above, calibration data may be calculated from the measurement data acquired along the trajectories 36, 37, 38 and 39. All that is required for this is a measurement at four acquisition angles, which may stand perpendicularly on one another in pairs, as shown.

FIG. 6 depicts an image dataset 40 calculated from the measurement data acquired by the measurement sequence 13. In the image dataset 40, a partition or slice of a patient 41 is imaged purely schematically, more specifically at the level of the liver 42.

A first, greater signal intensity, and consequently density of monounsaturated fatty acids, is present in this case in a first region 43 than in a second region 44. Corresponding image datasets, which are nothing other than parameter maps, may also be generated for the fat component, the water component, polyunsaturated fatty acids, and saturated fatty acids.

Although the disclosure has been illustrated and described in detail by the exemplary embodiments, the disclosure is not restricted by the disclosed examples and the person skilled in the art may derive other variations from this without departing from the scope of protection of the disclosure. It is therefore intended that the foregoing description be regarded as illustrative rather than limiting, and that it be understood that all equivalents and/or combinations of embodiments are intended to be included in this description.

It is to be understood that the elements and features recited in the appended claims may be combined in different ways to produce new claims that likewise fall within the scope of the present disclosure. Thus, whereas the dependent claims appended below depend from only a single independent or dependent claim, it is to be understood that these dependent claims may, alternatively, be made to depend in the alternative from any preceding or following claim, whether independent or dependent, and that such new combinations are to be understood as forming a part of the present specification.

1. A method for generating a magnetic resonance image dataset containing spectroscopic information from an in vivo measurement, the method comprising:
acquiring measurement data by a non-Cartesian k-space sampling scheme, wherein the measurement data is acquired at different echo times in order to measure a time-dependent signal waveform;
performing a measurement data correction in order to eliminate gradient errors;
regridding the measurement data to Cartesian coordinates;
Fourier-transforming the measurement data to produce an image dataset containing image elements;
determining at least one piece of spectroscopic information in a form of a fat component, at least one fatty acid component, or both the fat component and the at least one fatty acid component for at least some of the image elements using a model function applied to the signal waveform; and
generating at least one magnetic resonance image dataset in which one piece of the spectroscopic information is mapped in a spatially resolved manner.

2. The method of claim 1, wherein the measurement data is acquired using a radial sampling scheme.

3. The method of claim 2, wherein the measurement data is acquired from at least 200 different acquisition angles.

4. The method of claim 22, wherein the measurement data is acquired using a multigradient echo sequence.

5. The method of claim 1, wherein a 3-peak or a 4-peak model is used for determining the at least one fatty acid component.

6. The method of claim 1, wherein the fat component, the at least one fatty acid component, or both the fat component and the at least one fatty acid component are determined based on the measurement data processed as complex values.

7. The method of claim 1, wherein a frequency shift is taken into account in the determination of the fat component, the at least one fatty acid component, or both the fat component and the at least one fatty acid component.

8. The method of claim 1, wherein, in order to obtain calibration data for performing the measurement data correction, calibration measurement data is acquired using a same measurement sequence as for the acquisition of the measurement data of the image measurement dataset.

9. The method of claim 8, wherein a number of acquisition angles of the calibration measurement dataset is less than a number of acquisition angles of the image measurement dataset.

10. The method of claim 23, wherein precisely four acquisition angles are used in a radial sampling of the k-space during the acquisition of the calibration measurement data.

11. The method of claim 1, wherein a separate calibration value is determined for each echo signal in the measurement data.

12. The method of claim 1, wherein the measurement data at least partially images a liver.

13. A computer program product for a control device for controlling a data generation unit of a magnetic resonance system, wherein the computer program product, when executed, is configured to cause the data generation unit to:
acquire measurement data by a non-Cartesian k-space sampling scheme, wherein the measurement data is acquired at different echo times in order to measure a time-dependent signal waveform;
perform a measurement data correction in order to eliminate gradient errors;
regrid the measurement data to Cartesian coordinates;
Fourier-transform the measurement data to produce an image dataset containing image elements;
determine at least one piece of spectroscopic information in a form of a fat component, at least one fatty acid component, or both the fat component and the at least one fatty acid component for at least some of the image elements using a model function applied to the signal waveform; and
generate at least one magnetic resonance image dataset in which one piece of the spectroscopic information is mapped in a spatially resolved manner.

14. The computer program product of claim 13, wherein the data generation unit is an image generation unit.

15. A magnetic resonance system comprising:
a control device configured to:
acquire measurement data by a non-Cartesian k-space sampling scheme, wherein the measurement data is acquired at different echo times in order to measure a time-dependent signal waveform;
perform a measurement data correction in order to eliminate gradient errors;
regrid the measurement data to Cartesian coordinates;
Fourier-transform the measurement data to produce an image dataset containing image elements;
determine at least one piece of spectroscopic information in a form of a fat component, at least one fatty acid component, or both the fat component and the at least one fatty acid component for at least some of the image elements using a model function applied to the signal waveform; and
generate at least one magnetic resonance image dataset in which one piece of the spectroscopic information is mapped in a spatially resolved manner.
